(19) United States
(12) Patent Application Publication
(10) Pub. No.: US 2024/0335367 A1
(43) Pub. Date: Oct. 10, 2024

(54) METHOD OF TREATING OR PREVENTING PLAQUE ASSOCIATED DISEASES BY PHOSPHATASE

(71) Applicant: Biplab Kumar Malo, Reading, MA (US)

(72) Inventor: Biplab Kumar Malo, Reading, MA (US)

(21) Appl. No.: 18/296,445

(22) Filed: Apr. 6, 2023

(51) Int. Cl. A61K 8/66; A61Q 11/00

(52) U.S. Cl. CPC A61K 8/66; A61Q 11/00

(57) ABSTRACT

A method of treating and preventing disease includes administering an amount of alkaline phosphatase to a subject effective to prevent biofilm, plaque formation, calculus, and incipient dental caries in the subject's oral cavity; to prevent plaque formation in the blood vessels and brain; to prevent colonization of bacteria in skin, in the gut, and other parts of the body. The administered composition is a dentifrice, is ingestible, is injectable, or is topically applied. The method can cure any disease that is due to bacterial plaque formation that's surface attachment initiated by LTA, LPS and/or Tau protein.

### BACKGROUND OF THE INVENTION

The present invention relates to biofilm- and plaque-related disease and, more particularly, to a method of treating or preventing disease by administering alkaline phosphatase.

Millions of people suffer from dental caries, gingival disease, periodontal disease, atherosclerosis, Alzheimer's disease, and dementia. These conditions are believed to be due to biofilm and plaque formation initiated by the external layer of the gram positive bacterial cell wall which contain Lipoteichoic Acid (LTA) (Armstrong et al, 1958; Baddiley et al, 1989; Vinogradov et al, 2006; Olaf Schneewind and Dominique Missiakas, 2014) and Lipopolysaccharide (LPS) in gram negative bacterial cell walls. The early stage of plaque formation consists of mostly gram positive bacteria (Ratcliff Perry, 1986). Lipoteichoic acid (LTA) in gram-positive bacteria (Neuhaus et al, 2003) first adheres to a surface, such as the surface of a tooth, initiating plaque accumulation (Charles et al, 2005).

Zhang et al. showed that growing bacteria secrete LPS at a level in proportion to their growth phase (Zhang et al. 1998). Therefore, even a small fraction of the plaque left on the teeth after brushing could seed the inflammatory cascade due to the release of LPS from the gram-negative bacteria present in the plaque. In a recent publication, Malo et el. showed that IAP detoxify LTA and LPS (Malo J. et al, 2022) Therefore, the idea came across that IAP would definitely prevent the formation of plaques on teeth and other parts of the body including blood vessels, and brain.

Most currently used agents to prevent plaque are toxic upon ingestion, in the blood, and in the brain. Most of the current remedies and/or prophylactics use alcohol, fluoride, and positive ionic metals and cannot be used directly via injection into the blood system.

As can be seen, there is a need for a plaque prevention treatment that avoids toxic materials and can safely be directly applied on teeth surface, orally ingested, and injected in the blood circulation.

### SUMMARY OF THE INVENTION

In one aspect of the present invention, a method of treating and preventing disease comprises administering alkaline phosphatase to a subject in an amount effective to prevent biofilm and plaque formation in the subject's oral cavity, blood vessels, and brain; and to prevent colonization of oral bacteria to other parts of the body such as gut and brain.

In another aspect of the present invention, a composition for use in the method comprises the amount of alkaline phosphatase effective to prevent biofilm and plaque formation in the subject's oral cavity, blood vessels, and brain; and to prevent colonization of oral bacteria such as Fusobacterium nucleatum and Porphyromonas gingivalis to other parts of the body such as gut and brain.

The method described herein may cure any disease that is initiated by plaque formation due to Lipoteichoic acid (LTA) (Fedtke et al, 2007) and Lipopolysaccharide (LPS); and migration and colonization of oral bacteria to other parts of the body.

These and other features, aspects and advantages of the present invention will become better understood with reference to the following drawings, description, and claims.

### BRIEF DESCRIPTION OF THE DRAWINGS

FIG. 1 is a flow chart of illustrating the progression of disease that may occur due to biofilm forming in the oral cavity;

FIG. 2 is another flowchart of the progression of disease that may be due to biofilm forming in the oral cavity; and

FIG. 3 is a flow chart of a method of using alkaline phosphatase to treat and prevent disease according to an embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

As used herein, the term “subject” may refer to a human or an animal, including pets such as dogs and cats and barnyard animals such as pigs and cows, etc.

Broadly, one embodiment of the present invention is a method of treating and preventing various diseases by administering alkaline phosphatase to a subject. The treatment may be provided as a mouth wash and/or toothpaste or gel to prevent plaque formation in the oral cavity. Alternatively, it may be ingested and/or administered via injection to protect all the body's systems.

Alkaline phosphatase (AP), such as intestinal alkaline phosphatase (IAP), is a natural enzyme in the human body. It breaks down the phosphodiester bond in LTA, and LPS, thereby preventing formation of biofilm, plaque, incipient dental caries, and calculus on the teeth in the oral cavity, as well as in arteries and the brain. LTA, and LPS cannot adhere to a surface in presence of AP. The absence of biofilm due to the presence of AP, anywhere in the body, is believed to prevent associated diseases, such as dental caries, calculus, gingival disease, periodontal disease, atherosclerosis, Alzheimer's disease, and dementia, solving a problem at the initial stage of the disease process.

In some embodiments, the method comprises application of AP-containing toothpaste or gel in an amount effective to hydrolyze exposed LTA, thereby preventing adherence of gram-positive bacteria to the teeth surface. Thus, no dental caries, no calculus, and no gum disease result, preventing dissemination, migration, and colonization of oral bacteria directly to the blood.

In some embodiments, the method comprises application of AP-containing mouthwash in an amount effective to prevent LTA from adhering to the oral cavity, including the teeth surface, gingiva, and the tongue. Thus, no gum disease, calculus, and no caries results, preventing dissemination, migration, and colonization of oral bacteria directly to the blood.

In some embodiments, the method comprises administering AP as a tablet or capsule to be ingested by a subject in an amount effective to prevent intestinal alkaline phosphate deficiency (Malo et al, 2022) in the gut. Thus, LTA and LPS of bacterial cell walls are broken down, preventing entry of bacteria, LTA, and LPS into the blood stream via the gut. It may also prevent colonization of Fusobacterium nucleatum (Sun et al, 2019) in the gut as it prevents colonization on the tooth surface and on gingiva, preventing colon cancer associated with Fusobacterium nucleatum (Wu N. et al, 2022; Abed et al, 2020; and Sun et al, 2019).

In some embodiments, the method comprises administering AP by injection of alkaline phosphatase into the blood steam. The presence of alkaline phosphatase prevents bacteria having cell walls including LTA and LPS that may enter the blood stream, and Tau protein from adhering to any blood system vessel walls, including arteries, and glial cells and neuron in the brain.

TABLE 1
In vitro experiment with IAP doses in
mouthwash and results: after 72 hours

| Dose 1 | Dose 2 | Dose 3 | Dose 4 |
|---|---|---|---|
| 0 units | 10 units | 50 units | 100 units |
| IAP/ml | IAP/ml | IAP/ml | IAP/ml |
| mouthwash | mouthwash | mouthwash | mouthwash |
| Heavy | Moderate | Mild | No |
| bacterial | bacterial | bacterial | visible |
| growth | growth | growth | bacterial |
| | | | growth |
| Moderate | Mild | No | No |
| biofilm | biofilm | biofilm | biofilm |
| formation | formation | formation | formation |
| around | around | around | around |
| tooth | tooth | tooth | tooth |
| pH 7.5 | pH 8.0 | pH 8.5 | pH 9.0 |

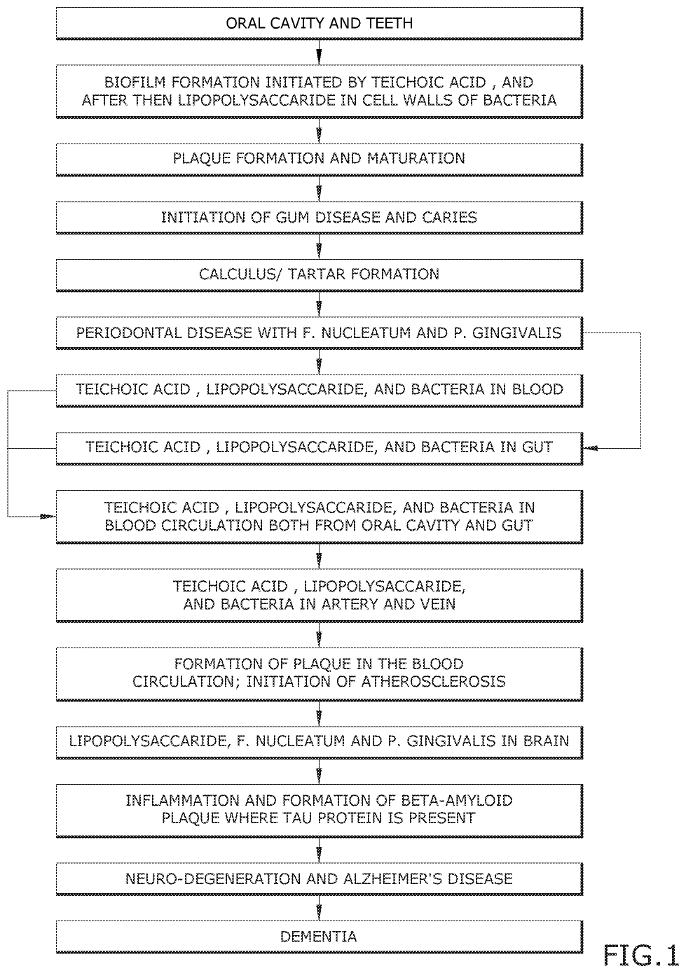

Referring to FIGS. 1 through 3, FIG. 1 illustrates an example of disease progression from biofilm formation in the oral cavity and teeth to dementia. The elimination of any step in the progression may prevent the occurrence of dementia, at least as it relates to biofilm and plaque formation. Teichoic acid and/or lipopolysaccharide in bacterial cell walls initiate biofilm formation. If the biofilm is not removed, plaque forms and matures, eventually initiating calculus formation, gum disease and caries. Plaque hardens into calculus and tartar forms, encouraging growth of Fusobacterium nucleatum and Porphyromonas gingivalis, eventually resulting in periodontal disease. Bacteria and/or teichoic acid and/or lipopolysaccharides released from bacterial cell walls enter the body via the blood and/or gut (i.e., migration to and colonization in the gut), forming plaque in blood vessels, eventually initiating atherosclerosis. Bacteria and/or teichoic acid and/or lipopolysaccharides that reach the brain cause inflammation and formation of beta-amyloid (Glenner, G. G., and Wong, C. W. 1984) plaque with associated tau protein (Ando et al, 2021). Tau protein contains a lot of phosphodiester bonds (Wang, Y., and Mandelkow, E., 2016) like LTA. Thus, LPS of Fusobacterium nucleatum, and Porphyromonas gingivalis can adhere to Tau protein without the presence of LTA. Eventually neurodegeneration and Alzheimer's Disease may accelerate (Ishida et al, 2017; Wu H. et al, 2022), producing dementia.

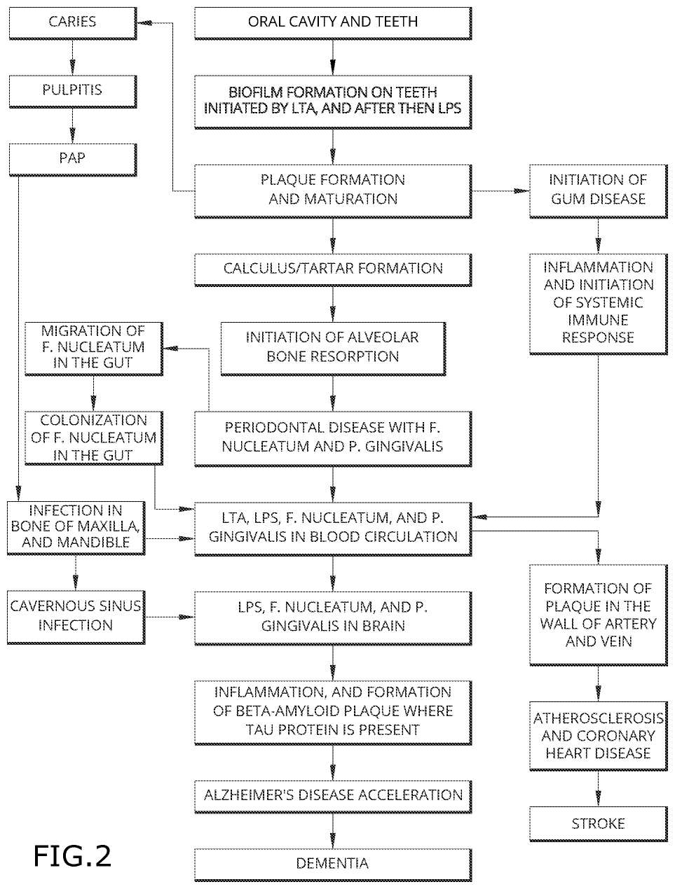

FIG. 2 further illustrates an example of disease progression from biofilm formation attributable first to LTA which allows the gram positive bacteria to adhere to the surface, after which LPS of gram negative bacteria adhere to form plaques. Gum disease is associated with inflammation and initiates a systemic immune response. Acid secretion by the bacteria in plaque causes erosion of enamel of the teeth in low pH, and leads to incipient caries, and caries. Caries may progress to Pulp necrosis, Periapical Pathology (PAP), and infection of the bone, including maxilla and the mandible. In some cases, a cavernous sinus infection may result, which can introduce LPS, Fusobacterium nucleatum and Porphyromonas gingivalis (Dominy et al, 2019) directly to the brain. The presence of LPS, Fusobacterium nucleatum and Porphyromonas gingivalis in the blood and/or brain may also result in formation of plaque in the arteries and/or veins, resulting in atherosclerosis, coronary heart disease, and in some cases, stroke.

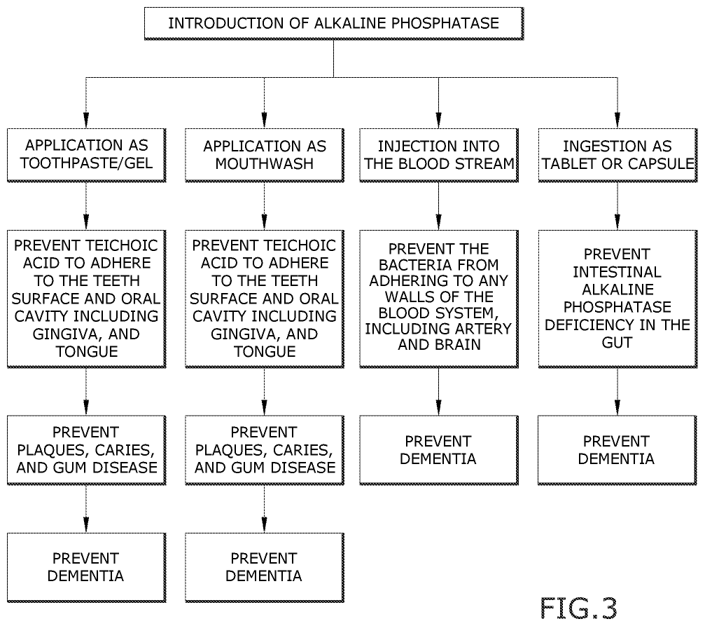

As shown in FIG. 3, the introduction of alkaline phosphatase, whether as a dentifrice such as a toothpaste or gel, a mouthwash, a tablet or capsule, or an intravenous composition is believed to prevent teichoic acid (TA), and lipopolysaccharide (LPS) of oral bacteria from adhering to surfaces in the oral cavity (teeth, gingiva, and tongue), the intestines, and surfaces of blood vessel walls, including in the brain. Without bacteria adhering within the body, dementia may be prevented either directly by breaking down LTA, and LPS or by preventing plaque accumulation, caries, and gum disease.

It should be understood, of course, that the foregoing relates to exemplary embodiments of the invention and that modifications may be made without departing from the spirit and scope of the invention as set forth in the following claims.

### REFERENCES

1. Abed et al. Colon Cancer-Associated Fusobacterium nucleatum May Originate From the Oral Cavity and Reach Colon Tumors via the Circulatory System. Front. Cell. Infect. Microbial. 2020; 10:400.
2. Ando et al. Alzheimer's Disease: Tau Pathology and Dysfunction of Endocytosis. Front. Mol. Neurosci. 2021; 13:583755.
3. Armstrong J J, Baddiley J, Buchanan J G, Carss B, Greenberg G R. Isolation and structure of ribitol phosphate derivatives (teichoic acids) from bacterial cell walls. J. Chem. Soc. 1958; 4344-4354.
4. Baddiley J.; Bacterial cell walls and membranes. Discovery of the teichoic acids. Bioessays. 1989 June; 10 (6): 207-10.
5. Charles V. Rice, and Jason R. Wickham. Heterogeneous Binding of Lipoteichoic Acid to the Surface of Titanium Dioxide as Determined with ³¹P Solid-State NMR Spectroscopy. J. Am. Chem. Soc., 2005; 127 (3): 856-857.
6. Dominy et al. Porphyromonas gingivalis in Alzheimer's disease brains: Evidence for disease causation and treatment with small-molecule inhibitors. Science Advances. 2019; 5 (1).
7. Fedtke et al. A Staphylococcus aureus ypfP mutant with strongly reduced lipoteichoic acid (LTA) content: LTA governs bacterial surface properties and autolysin activity. Molecular Microbiology. 2007; 65 (4): 1078-1091.
8. Glenner, G. G., and Wong, C. W. Alzheimer's disease: initial report of the purification and characterization of a novel cerebrovascular amyloid protein. Biochem. Biophys. Res. Commun. 1984; 120 (3): 885-890.
9. Ishida et al. Periodontitis induced by bacterial infection exacerbates features of Alzheimer's disease in transgenic mice. NPJ Aging Mech Dis. 2017; 3 (15).
10. Malo J. et al. Intestinal alkaline phosphatase deficiency increases the risk of diabetes. BMJ Open Diab Res Care. 2022; 10 (1).
11. Neuhaus, F. C., and J. Baddiley. A continuum of anionic charge: structures and functions of D-alanyl-teichoic acids in gram-positive bacteria. Microbiol. Mol. Biol. Rev. 2003; 67 (4): 686-723.
12. Olaf Schneewind and Dominique Missiakas. Lipoteichoic Acids, Phosphate-Containing Polymers in the Envelope of Gram-Positive Bacteria. J Bacteriol. 2014 March; 196 (6): 1133-1142.
13. Ratcliff Perry. Method for retarding formation of dental plaque. U.S. Pat. No. 4,925,656. 1986.
14. Sun et al. The role of Fusobacterium nucleatum in colorectal cancer: from carcinogenesis to clinical management. Chronic Dis Transl Med. 2019; 5 (3): 178-187.
15. Vinogradov E, Sadovskaya I, Li J, Jabbouri S. Structural elucidation of the extracellular and cell-wall teichoic acids of Staphylococcus aureus MN8m, a biofilm forming strain. Carbohydr Res. 2006 May 1; 341 (6): 738-43.
16. Wang, Y., and Mandelkow, E. Tau in physiology and pathology. Nat. Rev. Neurosci. 2016; 17 (1): 5-21.
17. Wu H. et al. The periodontal Pathogen Fusobacterium nucleatum Exacerbates Alzheimer's Pathogenesis via Specific Pathways. Front Aging Neurosci. 2022; 14:912709.
18. Wu N. et al. Fusobacterium nucleatum promotes colon cancer progression by changing the mucosal microbiota and colon transcriptome in a mouse model. World J gastroenterol. 2022; 28 (18): 1981-1995.
19. Zhang et al. Lipoprotein release by bacteria: potential factor in bacterial pathogenesis. Infection & Immunity. 1998; 66 (11): 5196-5201.

What is claimed is:

1. A method of treating and preventing disease, comprising:
administering alkaline phosphatase to a subject in an amount effective to prevent biofilm and plaque formation in the subject's oral cavity, blood vessels, skin, and brain; to break down LTA, and LPS in the gut and in blood circulation; and to prevent colonization of oral bacteria in other parts of the body.

2. The method of claim 1, wherein the alkaline phosphatase is administered by mouth or by injection.

3. The method of claim 1, wherein the alkaline phosphatase is contained in a carrier selected from the group consisting of: a dentifrice, a topical gel, a chewing gum, a mouthwash, a tablet, and a capsule.

4. The method of claim 1, wherein the disease is at least one selected from the group consisting of dental caries, calculus, gingival disease, periodontal disease, pulp necrosis, peri-apical pathology, oral-facial lesion, colon cancer associated with bacteria, atherosclerosis, blockage of coronary artery, Alzheimer's disease, and dementia.

5. A composition for use in the method of claim 1, comprising the amount of alkaline phosphatase effective to prevent biofilm and plaque formation in the subject's oral cavity, blood vessels, skin, and brain; to break down LTA and LPS in gut, blood circulation, glial cells, and neurons; and to prevent colonization of oral bacteria in another parts of the body.

6. The composition of claim 5, wherein the composition is a dentifrice, a toothpaste, a topical gel, or a mouthwash and the amount of alkaline phosphatase is further effective to hydrolyze exposed Lipoteichoic acid (LTA), and Lipopolysaccharide (LPS) in the subject's oral cavity, and skin surface of the body.

7. The composition of claim 5, wherein the composition is a tablet or capsule ingested containing an amount of alkaline phosphatase effective to prevent intestinal alkaline phosphate deficiency in the gut, and to prevent colonization of Fusobacterium nucleatum.

8. The composition of claim 5, wherein the composition is liquid, injected in the body, containing an amount of alkaline phosphatase effective to break down LTA and LPS in blood circulation, and in brain to prevent dementia associated with plaque formation of Fusobacterium nucleatum, Porphyromonas gingivalis and other bacteria.
